(19) United States
(12) Patent Application Publication
(10) Pub. No.: US 2022/0277842 A1
(43) Pub. Date: Sep. 1, 2022

(54) APPOINTMENT SYSTEM FOR UNFULFILLED APPOINTMENT OPTIMIZATION TO BE FILLED BY WAITING LIST PATIENTS

(71) Applicant: Ross Group of Companies Corp., Etobicoke (CA)

(72) Inventor: Wang-Piao Dumani ROSS, Toronto (CA)

(21) Appl. No.: 17/681,578

(22) Filed: Feb. 25, 2022

Related U.S. Application Data
(60) Provisional application No. 63/155,049, filed on Mar. 1, 2021.

(51) Int. Cl. G16H 40/20; G06Q 10/10; G16H 10/60; G16H 80/00; H04W 4/029

(52) U.S. Cl. CPC G16H 40/20; G06Q 10/1095; G16H 10/60; G16H 80/00; H04W 4/029

(57) ABSTRACT

Systems and methods for determining and filling unfulfilled appointments with stand-by patients. The unfulfilled appointment can be due to a no-show or inadequate preparation. The system can predict the unfulfilled appointment for a provider based on geography, patient calendar schedules, and other variables. In an example, the unfilled appointment is filled by a patient based on calendar availability and within a geographical proximity at the time of the unfulfilled appointment. The system can determine the unfulfilled appointment prior to a scheduled appointment, the length of time allotted to the scheduled appointment, prioritizing the eligible patients, alerting the eligible patients, receiving patient acceptance to fill the unfulfilled appointment, and selecting one of the patients to fill the unfulfilled appointment. The selected patient now has a sooner appointment than their originally scheduled appointment. The provider has now filled the potentially unfulfilled appointment by replacing with a new patient, saving resources.

### CROSS-REFERENCE

This application claims the benefit of priority to U.S. Provisional Patent Application No. 63/155,049 filed Mar. 1, 2021, the entire contents of which are incorporated by reference into the Detailed Description below.

### TECHNICAL FIELD

Example embodiments relate to appointment and scheduling software for providers, for example, systems and methods for unfulfilled appointment prediction and optimization to be filled by waiting list patients.

### BACKGROUND

A healthcare system needs to manage multiple appointments to effectively care for the patients. Optimal use of time is an important factor in improving successful outcomes when patients interact with the healthcare system. For example, patients of the medical imaging service industry are suffering from excessive wait times which stem from the foundation of having limited resources to meet a surplus of demand. In some countries, such as Canada, the delay to be seen is further enhanced by Canada having a public health care system. To mitigate the patient pain of excessive wait times, existing solutions from government and third parties include increasing provider operational hours and the development of centralized booking systems. Also, some governments are moving towards funding patients to be seen at private clinics. Some drawbacks to existing solutions include, and are not limited to: increased payroll and machine operation costs from longer service hours, the inertia of culture transformation that occurs from the replacement of existing booking systems and data integrations, and the large funding to cover patient exams at private clinics which is not aligned with a public healthcare mandate. In some cases, the public healthcare system has limited resources, a surplus of demand, and suffers from the high costs of unfulfilled appointments due to no-shows and inadequate preparation.

It would be advantageous to provide systems and methods for unfulfilled appointment prediction and optimization to be filled by patients who wish to schedule a sooner appointment.

### SUMMARY

Example embodiments relate to systems and methods for appointment and scheduling software for providers such as medical clinics. Example embodiments relate to systems and methods for unfulfilled appointment prediction and optimization to be filled by patients who wish to schedule a sooner appointment.

An example embodiment is a system having an appointment application for determining and filling unfulfilled appointments with stand-by patients. Example of unfulfilled appointments includes no-shows and inadequate preparation of requirements for the type of procedure for the appointment. In an example, the unfulfilled appointment is filled by a patient having calendar availability and within a geographical proximity at the time of the unfulfilled appointment. An unfulfilled appointment predictor module determines that there is a probability of an unfulfilled appointment prior to a scheduled appointment, for reasons of no-show or inadequate preparation. A patient selection module is configured to, based on the length of time allotted to the scheduled original appointment, patient records, geography, and patient calendar schedules, determine eligibility and priority of patients to fill the unfulfilled appointment. The appointment application alerts eligible patients through use of a frontend appointment application (e.g. mobile application or website), and patient acceptances to fill the unfulfilled appointment are received (patient rejections are also received). The appointment application outputs a selected patient from the patient acceptances to fill the unfulfilled appointment.

In an example, the appointment application includes to determine probabilities of unfulfilled appointments based on patient calendars. In an example, the unfulfilled appointment predictor module includes an unfulfilled appointment predictor model. In an example, the patient selection module includes a patient selection model.

An advantage is that the appointment application identifies a segment of patients who will receive their procedures sooner than initially scheduled, and, the appointment application fills booking slots from initially scheduled appointments which are no longer wasted due to no-shows and inadequate preparation.

An example embodiment is a method, which includes: receiving, from a respective device of at least one patient, calendar information of that patient from a calendar application of that device; determining a scheduled appointment as being an unfulfilled timeslot; determining at least one patient as being an eligible patient for filling the unfulfilled timeslot based on the calendar information of the respective device; sending a communication to the respective device of at least one of the eligible patients to confirm a respective patient acceptance to fill the unfulfilled timeslot; receiving the respective patient acceptance from at least one eligible patient to fill the unfulfilled timeslot; and scheduling at least one eligible patient having the respective patient acceptance to fill the unfulfilled timeslot.

In an example embodiment of any of the above, the method further includes: determining a priority of at least one of the eligible patients; and selecting, based on the priority, the at least one eligible patient to fill the unfulfilled timeslot for the scheduling.

In an example embodiment of any of the above, the priority is based on a respective patient electronic health record of the patient.

In an example embodiment of any of the above, the priority is based on a respective health status of the patient from the respective patient electronic health record.

In an example embodiment of any of the above, the priority of one of the patients increases when that patient had sent a previous patient acceptance and was not scheduled to fill a previous unfulfilled timeslot.

In an example embodiment of any of the above, the communication includes a deadline, wherein the receiving the respective patient acceptance is within the deadline in order for the scheduling to be authorized.

In an example embodiment of any of the above, the determining at least one patient as being the eligible patient is based on a time length of the unfulfilled timeslot and a respective time length of procedure for the eligible patient.

In an example embodiment of any of the above, the method further includes receiving, from the respective device of at least one patient, location information of that patient; wherein the determining at least one patient as being the eligible patient is further based on the location information of that patient.

In an example embodiment of any of the above, the device has a Global Positioning System, wherein the location information is obtained from the Global Positioning System of the respective device of the patient.

In an example embodiment of any of the above, the location information is obtained from the calendar application of the respective device of the patient.

In an example embodiment of any of the above, the location information is obtained from an input address of that patient.

In an example embodiment of any of the above, the unfulfilled timeslot is from a patient originally in the unfulfilled timeslot, wherein the determining the scheduled appointment as being the unfulfilled timeslot is determined from the calendar information of the calendar application of the respective device of that patient originally in the unfulfilled timeslot.

In an example embodiment of any of the above, the method further includes receiving, from the respective device of at least one patient, location information of that patient; wherein the unfulfilled timeslot is from a patient originally in the unfulfilled timeslot; wherein the determining the scheduled appointment as being the unfulfilled timeslot is based on the location information of that patient originally in the unfulfilled timeslot.

In an example embodiment of any of the above, the device has a Global Positioning System, wherein the location information is based from the Global Positioning System of the respective device of the patient.

In an example embodiment of any of the above, the location information is obtained from the calendar application of the respective device of the patient.

In an example embodiment of any of the above, the location information is obtained from an input address of that patient.

In an example embodiment of any of the above, the unfulfilled timeslot is from a patient originally in the unfulfilled timeslot, wherein the determining the schedule appointment as being the unfulfilled timeslot is based on the calendar information of that patient originally in the unfulfilled timeslot.

In an example embodiment of any of the above, the method further includes: receiving a respective pre-loaded payment from at least one patient; and applying the respective pre-loaded payment as a consequence to the scheduling at least one eligible patient.

In an example embodiment of any of the above, the unfulfilled timeslot is from a patient originally in the unfulfilled timeslot, wherein the determining the unfulfilled timeslot is due to a no-show of that patient originally in the unfulfilled timeslot.

In an example embodiment of any of the above, the unfulfilled timeslot is from a patient originally in the unfulfilled timeslot, wherein the determining the unfulfilled timeslot is due to inadequate preparation of that patient originally in the unfulfilled timeslot.

In an example embodiment of any of the above, the determining the unfulfilled timeslot is performed using an unfulfilled appointment predictor model.

In an example embodiment of any of the above, the method further includes selecting, using a patient selection model, the at least one eligible patient to fill the unfulfilled timeslot for the scheduling.

In an example embodiment of any of the above, the unfulfilled timeslot is from a patient originally in the unfulfilled timeslot, wherein the unfulfilled timeslot is due to a no-show of that patient originally in the unfulfilled timeslot.

In an example embodiment of any of the above, the scheduled appointment is stored in a provider electronic health record database.

In an example embodiment of any of the above, the method further includes receiving a request from at least one patient to be one of the eligible patients.

In an example embodiment of any of the above, the unfulfilled timeslot is from a patient originally in the unfulfilled timeslot, further comprising rescheduling the patient originally in the unfulfilled timeslot.

In an example embodiment of any of the above, each patient has a respective scheduled appointment, further comprising receiving from at least one patient a request to be the eligible patient for filling the unfulfilled timeslot.

In an example embodiment of any of the above, the request includes a referral from a referring medical doctor.

In an example embodiment of any of the above, the at least one eligible patient scheduled to fill the unfulfilled timeslot has a respective scheduled appointment with a first provider, wherein the unfulfilled timeslot is with a second provider different than the first provider.

In an example embodiment of any of the above, the unfulfilled timeslot is with a provider, wherein the method is performed by a processor operated by a third party from the provider, wherein the method is performed by the processor for multiple different providers including the provider.

Another example embodiment is a server, including a processor configured to perform the examples of the method.

Another example embodiment is a non-transitory memory containing instructions which, when executed by a processor, cause the processor to perform the examples of the method.

### BRIEF DESCRIPTION OF THE DRAWINGS

Reference will now be made, by way of example, to the accompanying drawings which show example embodiments, and in which:

FIG. 1 shows a schematic diagram of a system of appointment scheduling, according to an example embodiment;

FIG. 2 shows a detailed diagram of the system of FIG. 1, including a server and devices;

FIG. 3 shows a block diagram of the server in FIG. 2, according to an example embodiment;

FIG. 4 shows a block diagram of a device in FIG. 2, according to an example embodiment;

FIG. 5 illustrates a first schedule and a second schedule of a provider in accordance with an example embodiment;

FIG. 6 illustrates a method of appointment scheduling performed by the system, in accordance with an example embodiment;

FIG. 7A illustrates a signup process for the method of FIG. 6;

FIG. 7B illustrates a backend of the method of FIG. 6;

FIG. 7C illustrates a referring Medical Doctor (MD) process and an appointment filling process for the method of FIG. 6;

FIG. 8 illustrates a detailed example of the backend of the method of FIG. 6;

FIG. 9 illustrates an example of determining an unfulfilled appointment by way of a no-show, in accordance with an example embodiment;

FIG. 10 illustrates an example of determining an unfulfilled appointment by way of inadequate preparation, in accordance with an example embodiment;

FIG. 11 illustrates a first screen and a second screen of a device of a patient having the appointment scheduling performed by the server; and

FIG. 12 illustrates a method of appointment scheduling, in accordance with an example embodiment.

Similar reference numerals may have been used in different figures to denote similar components.

### DETAILED DESCRIPTION

Example embodiments relate to systems and methods for appointment and scheduling software for providers such as medical clinics. Example embodiments relate to systems and methods for unfulfilled appointment prediction and optimization to be filled by patients who wish to schedule a sooner appointment.

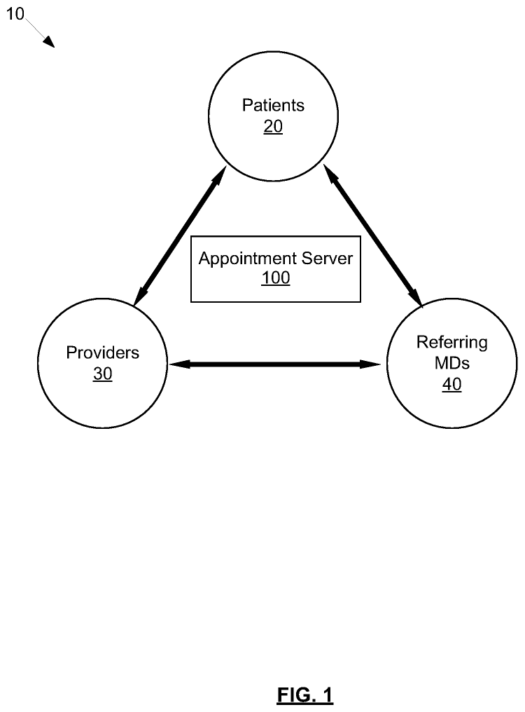

FIG. 1 illustrates a system 10 for appointment scheduling, according to an example embodiment. The system includes an appointment server 100, one or more patients 20, one or more providers 30, and one or more referring Medical Doctors (MDs) 40. Any reference to any singular entity can equally apply to a multiplicity of the same entity, and vice versa. An example of the provider 30 is a clinic such as a medical clinic. The types of appointments can include procedures such as medical procedures for the patients 20. In an example, each of the patients 20 may have a scheduled appointment with one of the providers 30. In some cases, the originally schedule appointment can be a long wait, and some patients 20 await the time of the original scheduled appointment to complete the procedure. In an example, the appointment server 100 is a third party to the providers 30 and the referring MDs 40. In another example, the appointment server 100 is commonly operated by one of the providers 30 or by a central provider such as the government.

In an example, the appointment server 100 is configured to perform a method of appointment scheduling as described in greater detail herein. In an example, at least one of the patients 20 may wish to schedule a sooner appointment than their originally scheduled appointment. In an example, the appointment server 100 determines that one of the scheduled appointments is an unfulfilled timeslot, for example due to a no-show or insufficient preparation. In an example, the appointment server 100 determines one or more eligible patients 20 to fill the unfulfilled timeslot, and sends a communication to those patients. In an example, the appointment server 100 receives patient acceptances and selects at least one of the patient acceptances to fill the unfulfilled timeslot. In an example, the referring MD 40 is a practitioner who has referred a patient 20 whom has a scheduled appointment to the appointment server 100 when that patient 20 wishes to sign up for being eligible for a sooner appointment. In some jurisdictions, such as Canada, the referring MD 40 is required (e.g. mandatory) to issue a referral in order for a patient 20 to obtain an appointment with the provider 30 to perform the procedure.

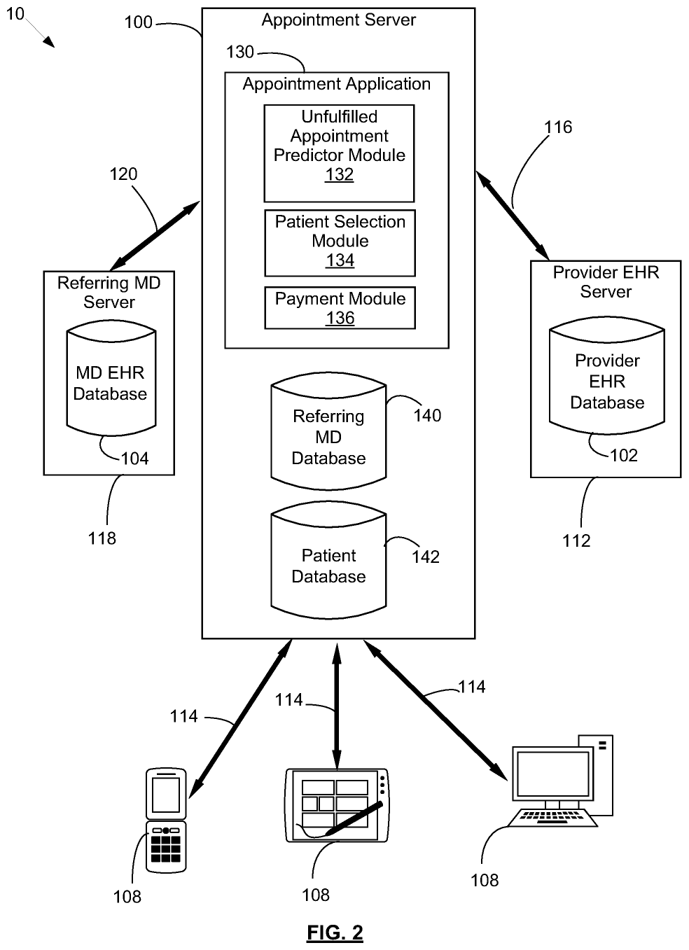

FIG. 2 is a detailed example of the system 10, in accordance with an example embodiment. The system 10 may include the appointment server 100, a referring MD server 118, a provider electronic health record (EHR) server 112, and one or more devices 108.

The provider EHR server 112 includes a provider EHR database 102 which stores EHR of patients 20. In an example, the EHR of the patients 20 are particular to that particular provider 30.

The referring MD server 118 includes a MD EHR database 104 which stores EHR of patients 20. In an example, the EHR of the patients 20 are particular to that particular referring MD 40. In another example, there is no referring MD server 118 and the referring MD 40 accesses the appointment server 100 using one of the devices 108.

In an example, the appointment server 100 is a third party the provider EHR servers 112 and the MD servers 118. In another example, the appointment server 100 is a first party server (commonly owned and operated, and may be integrated) with the provider EHR servers 112 or the MD servers 118 or by a central provider server operated by the government.

The appointment server 100 is configured to perform a method of appointment scheduling, for example to identify a scheduled appointment as being an unfulfilled timeslot, and finding eligible patients 20 to fill the unfulfilled timeslot. In an example, the appointment server 100 includes an appointment application 130, a referring MD database, and a patient database 142. In an example, the referring MD database 140 is for storing information of the referring MDs 40 or patient information from the MD EHR database 104. In an example, the patient database 142 is for storing patient profiles of patients 20. The patient profiles can be for those patient 20 that wish to register with the appointment server 100. The patient profiles can be for those patients 20 referred to the appointment server 100 by the referring MDs 40 or the referring MD server 118. The patient profiles can include sufficient information to access the correct patient record from the provider EHR database 102 or the MD EHR database 104. The patient profiles in the patient database 142 can include the EHR itself of the patients 20, collected from the various sources such as the provider EHR database 102, the MD EHR database 104, or from the device 108 of the patient 20.

The appointment application 130 can include an unfulfilled appointment predictor module 132, a patient selection module 134, and a payment module 136. The payment module 136 is used to process payments. For example, the payment module 136 can process payments for the procedures at the time of the scheduled appointment, usually after the procedure is completed but can be before the procedure in some instances. In another example, the payment module 136 can receive a pre-payment from a patient 20 who wishes to be eligible to schedule a sooner appointment, and the payment module 136 completes the pre-payment at the time of the sooner appointment (before or after the procedure).

In an example, appointment application 130 can include machine learning models and artificial intelligence (AI). For example, the unfulfilled appointment predictor module 132 can includes an unfulfilled appointment predictor model and the patient selection module 134 includes a patient selection model. For example, the unfulfilled appointment predictor model can use an artificial neural network (also referred to simply as a “neural network”) to predict a scheduled appointment as being an unfilled timeslot. For example, the patient selection model can use a neural network to identify eligible patients 20 to fill the unfilled timeslot, and prioritize the eligible patients 20 to fill the unfilled timeslot.

In some examples, the appointment server 100 can be a cloud server. The appointment server 100 in such an example is a cloud platform, with which the appointments can be managed and filled on the cloud platform. In an example, the provider EHR server 112 is a cloud server. In an example, the referring MD server 118 is a cloud server.

The device 108 can be an electronic device or user equipment for interacting with the user and for communicating with the appointment server 100. In examples, the device 108 can be used by the patients 20, the providers 30, and the referring MDs 40 to access the appointment server 100. The device 108 can be a desktop, a laptop, or a mobile communication device, such as a smart phone or a tablet. In some examples, the device 108 can include information of the patient 20, such as location information and calendar information. The device 108 can transmit the location information and the calendar information to the appointment server 100 via the communication link 114. The device can transmit a patient profile of the patient 20 to the appointment server.

The appointment server 100 can receive the location information, calendar information, patient profile, and other information, and store to the patient database 142. The unfulfilled appointment predictor module 132 can infer, from the received location information, calendar information, patient profile, and other information: that a scheduled appointment will be an unfulfilled timeslot. The unfulfilled timeslot can be due to a predicted no-show in an example. The patient selection module 134 can generate, from the location information, calendar information, patient profile, and other information: which patients 20 are eligible to fill the unfulfilled timeslot and a priority of those eligible patients 20.

The communication links 106, 116, 114 can be provided by a wireless or wired communications network.

Referring to the unfulfilled appointment predictor model and the patient selection model, a respective machine learning model can be implemented by a neural network running on a computing platform such as the appointment server 100. Neural networks will be briefly described in general terms. A neural network can include multiple layers of neurons, each neuron receiving inputs from a previous layer, applying a set of weights to the inputs, and combining these weighted inputs to generate an output, which can in turn be provided as input to one or more neurons of a subsequent layer.

A layer of neurons uses filters to define the relationship between the outputs of the neurons of the previous layer and the outputs of the neurons of the current layer. A layer of the neural network receives a data input, usually in the form of a data array of known dimensions. By applying the set of filters (layers) to the data input, each layer generates a data output, which is typically a data array having known dimensions. A filter comprises a set of weights (also called parameters).

The machine learning model is trained to infer i) the prediction of an unfulfilled appointment and ii) patient eligibility and priority to fill the unfulfilled appointment. In the example of a neural network, training a neural network involves learning or determining the appropriate weight values at different weight locations throughout the network. After being optimally trained to perform a given inference task, the weights of the neural network will not all contribute equally to the final inference outputs: some weights will have high value due to their high contribution, while other weights will have low value due to their low contribution. If the weights are not properly trained (e.g., high value weights are misplaced or miscalibrated by training), then the trained network will perform with less accuracy. In the appointment application 130, the machine learning model can be trained by a suitable set of training data to determine appropriate weights. The training data may include a labeled set of inputs that can be based on simulated or actual scenarios. The trained machine learning model can be used to create and apply models for performing the inference tasks.

In some examples, the trained machine learning model includes a support vector machine (SVM) or linear regression.

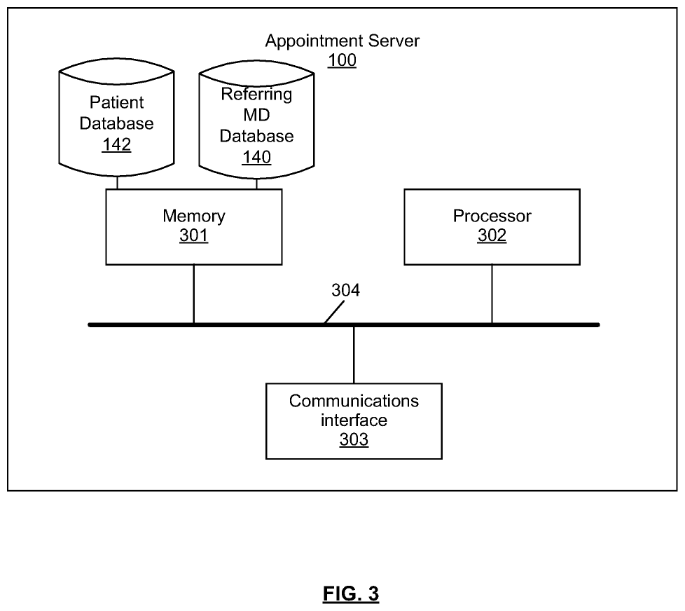

FIG. 3 is a schematic diagram of a hardware structure of the appointment server 100 according to an example embodiment. The appointment server 100 shown in FIG. 3 includes a memory 301, a processor 302, a communications interface 303, and a bus 304. A communication connection is implemented between the memory 301, the processor 302, and the communications interface 303 by using the bus 304.

The processor 302 is configured to perform, when the program stored in the memory 301 is executed by the processor 302, steps of the method of appointment scheduling as described herein.

The memory 301 can be a read-only memory (Read Only Memory, ROM), a static storage device, a dynamic storage device, or a random access memory (Random Access Memory, RAM). The memory 301 may store a program. The memory 301 can be a non-transitory memory. The memory 301 can be external or removable in some examples. In an example, the memory 301 includes the patient database 142 and the referring MD database 140. In an example, the memory 301 includes the appointment application 130.

The processor 302 can be a general central processing unit (Central Processing Unit, CPU), a microprocessor, an application-specific integrated circuit (Application Specific Integrated Circuit, ASIC), a graphics processing unit (graphics processing unit, GPU), or one or more integrated circuits.

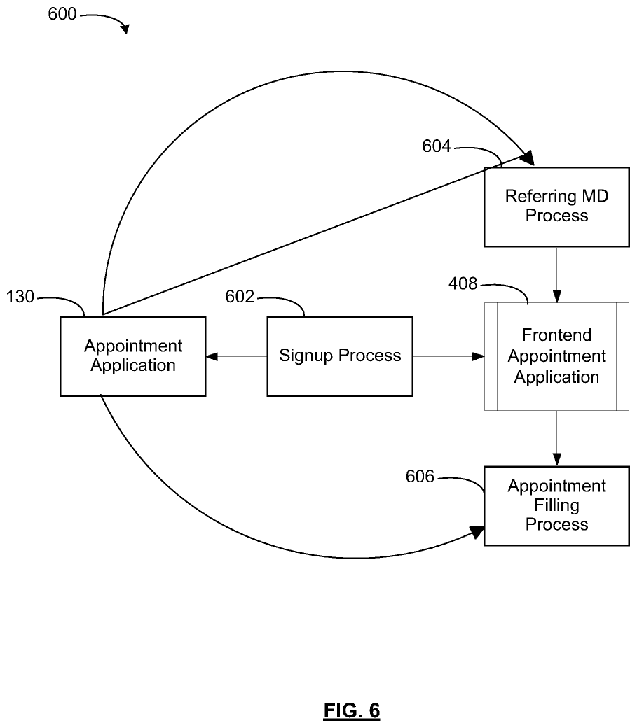

In addition, the processor 302 may be an integrated circuit chip with a signal processing capability. In an implementation process, steps of the method of appointment scheduling as described herein can be performed by an integrated logical circuit in a form of hardware or by an instruction in a form of software in the processor 302. In addition, the processor 302 can be a general purpose processor, a digital signal processor (Digital Signal Processor, DSP), an application-specific integrated circuit (ASIC), a field programmable gate array (Field Programmable Gate Array, FPGA) or another programmable logic device, a discrete gate or a transistor logic device, or a discrete hardware assembly. The processor 302 can implement or execute the methods, steps, and logical block diagrams that are described in example embodiments. The general purpose processor can be a microprocessor, or the processor may be any conventional processor or the like. The steps of the method disclosed with reference to the example embodiments may be directly performed by a hardware decoding processor, or may be performed by using a combination of hardware in the decoding processor and a software module. The software module may be located in a mature storage medium in the art, such as a random access memory, a flash memory, a read-only memory, a programmable read-only memory, an electrically erasable programmable memory, or a register. The storage medium is located in the memory 301. The processor 302 reads information from the memory 301, and completes, by using hardware in the processor 302, the steps of method 600 as detailed in FIG. 6.

The communications interface 303 implements communication between appointment server 100 and another device or communications network by using a transceiver apparatus, for example, including but not limited to a transceiver. For example, the training data may be obtained by using the communications interface 303.

The bus 304 may include a path that transfers information between all the components of the appointment server 100.

It should be noted that, although only the memory 301, the processor 302, and the communications interface 303 are shown in the appointment server 100 in FIG. 3, in a specific implementation process, a person skilled in the art should understand that the appointment server 100 may further include other components that are necessary for implementing normal running In addition, based on specific needs, a person skilled in the art should understand that the appointment server 100 may further include hardware components that implement other additional functions. In addition, a person skilled in the art should understand that the appointment server 100 may include only a component required for implementing the embodiments, without a need to include all the components shown in FIG. 3.

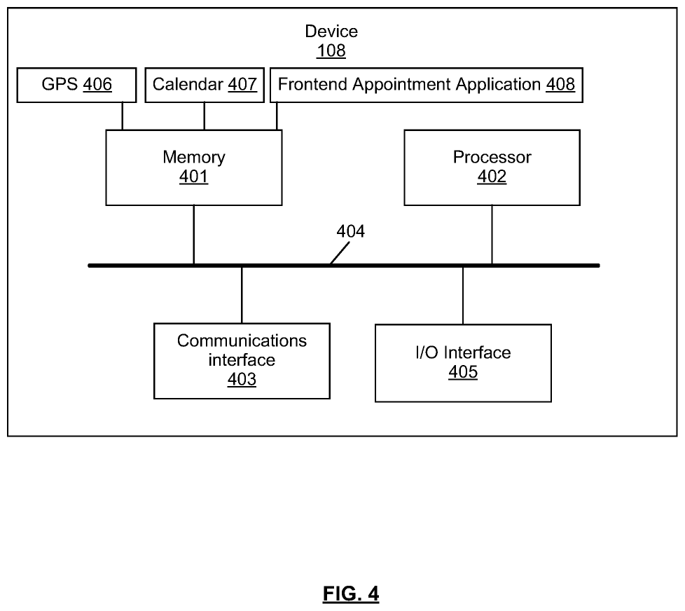

FIG. 4 is a schematic diagram of a hardware structure of the device 108 according to an example embodiment. The device 108 shown in FIG. 4 includes a memory 401, a processor 402, a communications interface 403, and a bus 404. A communication connection is implemented between the memory 401, the processor 402, and the communications interface 403 by using the bus 404.

The processor 402 and the communications interface 403 are configured to perform, when the program stored in the memory 401 is executed by the processor 402, steps of method 600 (e.g., alone or in combination with the appointment server 100).

The memory 401 can be a read-only memory (Read Only Memory, ROM), a static storage device, a dynamic storage device, or a random access memory (Random Access Memory, RAM). The memory 401 may store a program. The memory 401 can be a non-transitory memory. The memory 401 stores various applications that are resident on the device 108. In an example, the memory 401 stores a Global Positioning System (GPS) application 406 for interfacing with a GPS module of the device 108. In an example, the memory 401 stores a calendar application 407 for storing, managing, and maintaining the schedules of the user of the device 108, i.e. the patient 20. In an example, the memory 401 stores a frontend appointment application 408. In an example, the frontend appointment application 408 is a dedicated application for interfacing with the appointment server 100. In another example, the frontend appointment application 408 is a web browser or Internet access application in which the appointment server 100 is accessed by way of an address, portal, etc. In such an example, the appointment server 100 performs the logic of the method of appointment scheduling and the frontend appointment application 408 is used provide a frontend interface between the patient 20 and the appointment server 100.

In an example, information such as the calendar information, the location information, and other information can be sent to the appointment server 100 in order for the appointment server 100 to perform the method of appointment scheduling. For example, when patient 20 has a scheduled appointment and the location information or the calendar information (or both) suggests that the patient 20 will not be able to arrive at the scheduled appointment, the appointment application 130 can infer that the scheduled appointment is a no-show and has an unfulfilled timeslot. Other information can be used for the inference by the appointment application 130, such as weather, traffic, etc.

In another example, when a patient 20 requests to be eligible to fill an unfulfilled timeslot, the location information or the calendar information (or both) can be used by the appointment application 130 to determine that the patient 20 is an eligible patient. For example, the patient 20 may not be available to fill the unfulfilled timeslot due to another appointment for the unfulfilled timeslot, as indicated by their calendar application 407. In some instances, the patient 20 may not be in the geographical vicinity of the provider 30 at the time of the unfulfilled timeslot, in view of their location information or the calendar information. Other information can be used for the inference by the appointment application 130 to determine that the patient 20 is eligible, for example when the patient 20 was previously eligible but not selected by the appointment application 130 for a previous unfilled appointment. In such a scenario, the patient 20 can be deemed eligible and have a higher priority. Another example of information for the inference is patient condition, in which a patient 20 with a worsened condition may be deemed eligible and have a higher priority.

In an example, the likelihood of unfulfilled appointments can be incorporated into the frontend appointment application 408 to display the probabilities (e.g. hourly or weekly) of unfulfilled appointments.

The device 108 can include an operating system (not shown), for example Android™, iOS™, Microsoft Windows™, or other appropriate operation systems. A user can download the application to the device 108. In some examples, the device 108 can include an input/output (IO) interface 405 which is configured to interface with the user, for example via a touch screen, keyboard, buttons, microphone, speaker, etc.

The processor 402 can be a general central processing unit (Central Processing Unit, CPU), a microprocessor, an application-specific integrated circuit (Application Specific Integrated Circuit, ASIC), a graphics processing unit (graphics processing unit, GPU), or one or more integrated circuits.

In addition, the processor 402 may be an integrated circuit chip with a signal processing capability. In an implementation process, steps of method 600 can be performed by an integrated logical circuit in a form of hardware or by an instruction in a form of software in the processor 402. In addition, the processor 402 can be a general purpose processor, a digital signal processor (Digital Signal Processor, DSP), an application-specific integrated circuit (ASIC), a field programmable gate array (Field Programmable Gate Array, FPGA) or another programmable logic device, a discrete gate or a transistor logic device, or a discrete hardware assembly. The processor 402 can implement or execute the methods, steps, and logical block diagrams that are described in the example embodiments. The general purpose processor can be a microprocessor, or the processor may be any conventional processor or the like. The steps of the appointment scheduling method disclosed with reference to the example embodiments may be directly performed by a hardware decoding processor, or may be performed by using a combination of hardware in the decoding processor and a software module. The software module may be located in a mature storage medium in the art, such as a random access memory, a flash memory, a read-only memory, a programmable read-only memory, an electrically erasable programmable memory, or a register. The storage medium is located in the memory 401. The processor 402 reads information from the memory 401, and completes, by using hardware in the processor 402, the steps of method 600.

The communications interface 403 implements communication between device 108 and another device or communications network by using a transceiver apparatus, for example, including but not limited to a transceiver. For example, the training data may be obtained by using the communications interface 403. The device 108 is also configured to communicate with the appointment server 100 via one or more communication channels of a communication network, for example, a wireless communication network, the Internet, or other communication systems.

The bus 404 may include a path that transfers information between all the components of the device 108.

It should be noted that, although only the memory, the processor, and the communications interface are shown in the device 108 in FIG. 4, in a specific implementation process, a person skilled in the art should understand that the device 108 may further include other components that are necessary for implementing normal running In addition, based on specific needs, a person skilled in the art should understand that the device 108 may further include hardware components that implement other additional functions. In addition, a person skilled in the art should understand that the device 108 may include only a component required for implementing the embodiments, without a need to include all the components shown in FIG. 4.

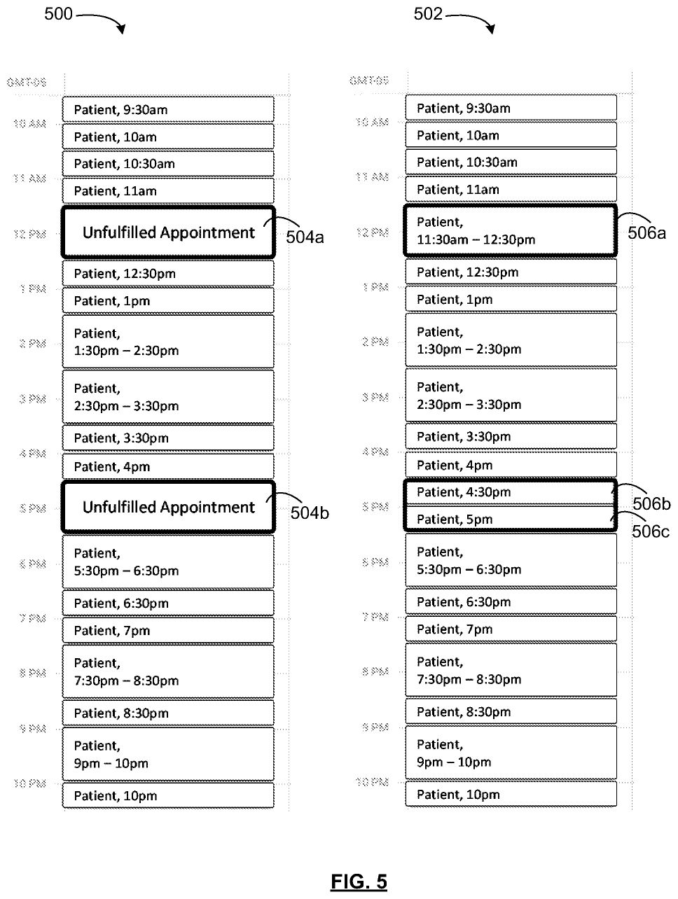

FIG. 5 illustrates a first schedule 500 and a second schedule 502 of the provider 30, in accordance with an example embodiment. In an example, the appointment server 100 determines at least one unfulfilled appointment of the first schedule 500 and fills the unfulfilled timeslot of the scheduled appointment with another patient 20. In an example, the first schedule 500 represents one day and the second schedule 502 represents the same one day after being filled by the appointment server 100. As illustrated in the first schedule 500, the first schedule 500 can include a plurality of scheduled appointments. Each of the scheduled appointments have a time length, for example 30 minutes or 1 hour in the present examples. As illustrated by the first schedule 500, at least one of the scheduled appointments can be determined by appointment application 130 as being an unfulfilled timeslot. For example, the unfulfilled appointment predictor module 132 can predict that a scheduled appointment is or is probably an unfulfilled timeslot. In another example, the appointment application 130 determines that the scheduled appointment is an unfulfilled timeslot due to inadequate preparation, e.g. inadequate preparation by the patient 20. In another example, the appointment application 130 simply receives a message from the provider 30, the provider EHR server 112, or the patient 20 (via the device 108) that the patient 20 will not be attending. As shown in FIG. 5, the appointment application 130 can determine that more than one scheduled appointment has an unfulfilled timeslot, for example two unfulfilled timeslots are shown in the first schedule 500 as first unfulfilled timeslot 504a at 11:30 am and second unfulfilled timeslot 504b at 4:30 pm.

The second schedule 502 illustrates performance of the appointment application 130 at a time after the first schedule 500, for example after determining that there is at least one unfulfilled timeslot. In an example, the appointment application 130 fills the two unfulfilled timeslots with patients 20 that wish to have a sooner appointment than their originally scheduled appointments. As shown, the appointment application 130 fills the first unfulfilled timeslot 504a with a first scheduled appointment 506a. The selection of the patient 20 to fill the first scheduled appointment 506a is performed by the appointment application 130. As shown in FIG. 5, the first scheduled appointment 506a is filled at 11:30 am and the time length is 1 hour. Similarly, the appointment application fills the second unfulfilled timeslot 504b in the 1 hour unfulfilled timeslot with two appointments: a second scheduled appointment 506b which is 30 minutes long and a third scheduled appointment 506c which is 30 minutes long. The second scheduled appointment 506b is at 4:30 pm and the third scheduled appointment 506c is at 5:00 pm. The patients 20 each had their originally scheduled appointments at a later time, and now have a sooner appointment as illustrated in the second schedule 502. In some examples, the patients 20 had their originally scheduled appointments at a different provider 30 than the provider 30 having the second schedule 502. In other examples, the patients 20 had their originally scheduled appointments at the same provider 30 having the second schedule 502.

Note that a particular provider 30 may have more than one schedule in parallel, for example each representing a different procedure room, patient capacity, or practitioner, etc.

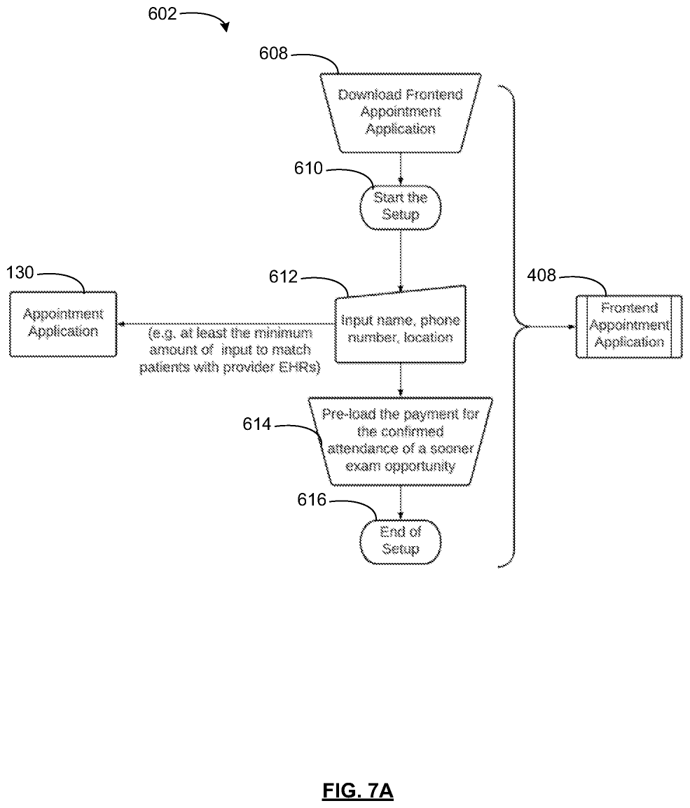

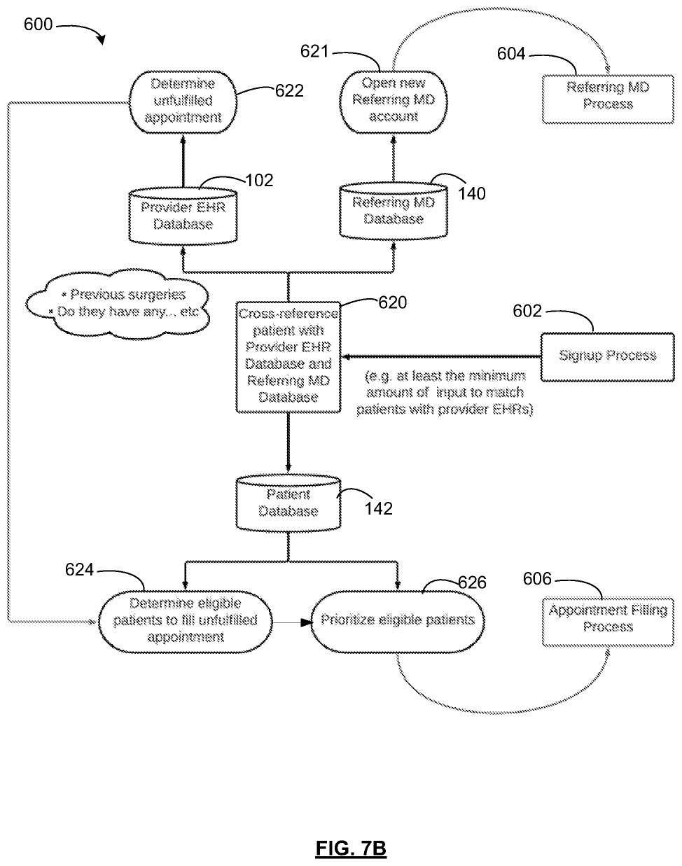

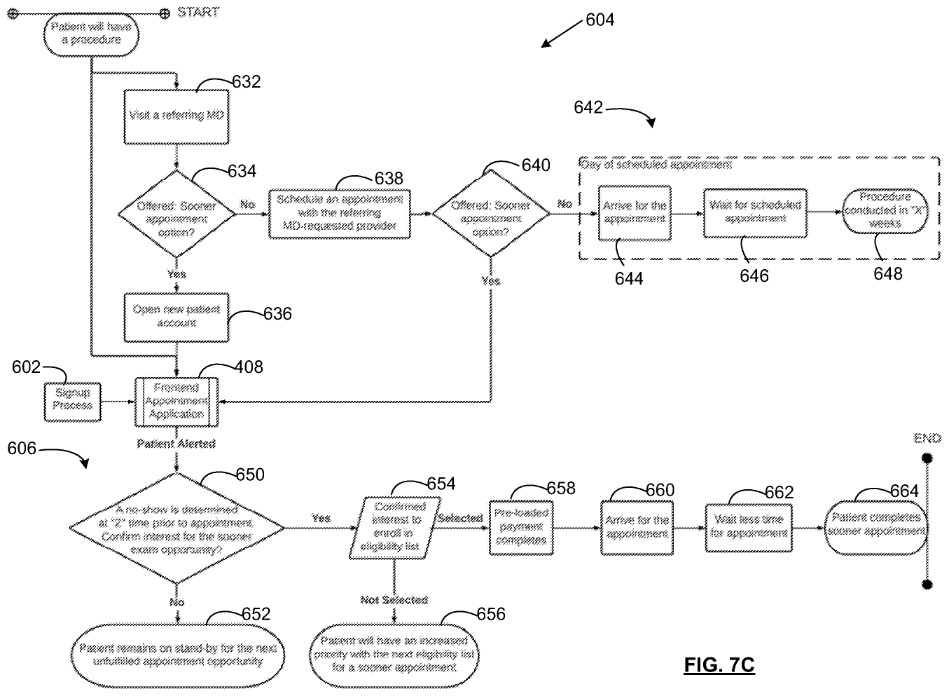

FIG. 6 illustrates a method 600 of appointment scheduling performed by the system 10, in accordance with an example embodiment. The method 600 includes a signup process 602, a referring MD process 604, and an appointment filling process 606. As illustrated in detail in FIG. 7B, a backend of the method 600 can be performed by the appointment application 130.

FIG. 7A illustrates the signup process 602 for the method 600 of appointment scheduling, in accordance with an example embodiment. For example, the method 600 can be performed by the appointment server 100 through the device 108 of the patient 20. In an example, the patient 20 has a scheduled appointment and wishes to schedule a sooner appointment when there is an unfulfilled timeslot. At step 608, the patient 20 downloads the frontend appointment application 408. At step 610, the patient 20 starts the setup of the frontend appointment application 408. In some examples, the setup of the frontend appointment application 408 includes granting permissions to the device 108, such as the GPS application 406 and the calendar application 407. For example, location information obtained from the GPS application can be sent from the device 108 to the appointment server 100. Similarly, calendar information obtained from the calendar application 407 can be sent from the device 108 to the appointment server. The device 108 can transmit the location information and the calendar information continuously, intermittently, at machine-learned time periods, after set triggers, etc. At step 612, the patient 20 inputs information of the patient profile. In an example, the patient profile includes name, phone number, and location (e.g. home, work and/or school). The patient can input the applicable address of the location. In an example, the patient profile includes at least the minimum amount of input required to match the patient 20 with the provider EHR database 102 or the MD EHR database 104. In another example, a record number for the patient 20 of the provider EHR database 102 or the MD EHR database 104 is input into the device 108.

At step 614, the patient 20 pre-loads the payment when the patient wishes to be eligible for a sooner appointment opportunity. In an example, the pre-loading of the payment is performed by the payment module 136. At step 616, the setup ends. In an example, the appointment application 130 receives the information from the signup process 602. The appointment application 130 can synchronize or retrieve the EHR of the patient 20 from the MD EHR database 104 or the provider EHR database 102. Therefore, the medical history of the patient 20 is known by the appointment application 130. The appointment application 130 can create a new patient profile (which can include the EHR) for that patient 20 into the patient database 142.

FIG. 7B illustrates a backend of the method 600 of appointment scheduling performed by the appointment application 130, in accordance with an example embodiment. The appointment application 130 receives the input information or patient profile generated by the signup process 602. At step 620, the appointment application 130 cross-references the received input information or patient profile with the provider EHR database 102 so that the correct patient 20 is identified and that the appropriate EHR of the patient 20 can be retrieved. At step 620, the appointment application 130 also cross-references the received input information or patient profile with the referring MD database 140, to see if the referring MD 40 is already registered with the appointment server 100. If the referring MD database 140 already includes the information of the referring MD 40, then the information of the referring MD 40 is retrieved from the referring MD database 140. If the referring MD database 140 does not include the information of the referring MD 40, then at step 621 the appointment application 130 opens a new referring MD account. The referring MD 40 having the new referring MD account can then refer future patients 20 to the appointment server via the referring MD process 604, shown in greater detail in FIG. 7C.

At step 622, the appointment application 130 analyzes the schedule of the provider EHR database 102. The appointment application 130 determines an unfulfilled appointment of the schedule of the provider EHR database 102. In an example, the unfulfilled appointment can be due to determining (receiving or predicting) a patient no-show for the unfulfilled timeslot. In another example, the unfulfilled appointment is due to inadequate preparation.

The appointment application 130 also maintains the patient database 142 for the patients 20 that wish to have a sooner appointment. At step 624, the appointment application 130 determines which of those patients 20 are eligible patients to fill the unfulfilled timeslot. At step 626, the appointment application 130 prioritizes the eligible patients, based on factors such as location information, calendar information, probability of attendance, medical history, and whether that patient 20 had previously accepted but was not selected to fill a previous unfulfilled timeslot.

FIG. 7C illustrates a detailed example of the referring MD process 604 and the appointment filling process 606, from the perspective of the patient 20 and the frontend appointment application 408, in accordance with an example embodiment. For the referring MD process 604, a referring MD account may be opened at step 621 (FIG. 7B). In such an instance, the referring MD 40 is registered with the referring MD database 140, and wishes to have their patients 20 considered to have sooner appointments than their originally scheduled appointments.

At step 630, a patient 20 requires a procedure (e.g. medical exam). At step 632, the patient 20 visits a referring MD 40. At step 634, the referring MD 40 offers to the patient 20 an option to sign up for a sooner appointment, in case there is an unfulfilled appointment. At step 636, the patient 20 opens a new patient account, for example using the signup process 602 (FIG. 7A). In some examples, the patient 20 requires a procedure and directly signs up for a sooner exam through the frontend appointment application 408, for example, when both the patient 20 and the referring MD 40 are already registered with the appointment server 100.

At step 638, the MD 40 schedules an appointment for the patient 20, i.e., this is an originally scheduled appointment. At step 640, the referring MD 40 offers to the patient 20 the opportunity for a sooner appointment than the originally scheduled appointment. The patient 20 can accept the offer by using the frontend appointment application 408, and performing the signup process 602, etc. Process 642 illustrates the day of the originally scheduled appointment. For example, process 642 occurs when the patient 20 either chose not to accept the opportunity for the sooner appointment, or the patient 20 accepted and the appointment application 130 was unable to successfully schedule a sooner appointment. At step 644, the patient 20 arrives at the appointment. At step 646, the patient 20 waits for the procedure (e.g. exam). At step 648, the procedure is performed on the patient 20, and is completed in the originally scheduled “X” weeks from making the appointment. In some instances, the patient 20 will have been allotted a sooner appointment by the appointment application 130.

Still referring to FIG. 7C, the appointment filling process 606 will now be described from the perspective of the patient 20 and the frontend appointment application 408. A no-show is determined by the appointment application 130 at “Z” time prior to appointment. The patient 20 is alerted through the frontend appointment application 408. In an example, the patient 20 has a deadline to send a patient acceptance (or patent rejection) to the alert. In an example, the deadline is only a short period of time, such as 5 minutes or 1 hour. In another example, the deadline is a longer period such as from 1 day to 1 week. At step 650, in response to the alert, the patient 20 is to confirm interest in the sooner appointment. If the patient 20 does not accept (i.e., “No”), at step 652 the patient 20 remains on stand-by for the next unfulfilled appointment opportunity. If the patient 20 does accept (i.e., “Yes”), then at step 654 the frontend appointment application 408 sends the patient acceptance to the appointment application 130. At this stage, the appointment application 130 has a priority of the patients 20 (i.e., priority list) and the appointment application 130 application selects at least one patient 20 from the patients 20 that had sent a patient acceptance. For example, at least one of the patients having the highest priority from the priority list are selected. Note that more than one patient 20 can be selected, e.g. two procedures of half hour time length can fill a one hour unfulfilled timeslot. Example timeslots can be longer or shorter in other examples. When the patient 20 of interest is not selected (i.e., “Not Selected”), the patient 20 can be given an increased priority by the patient selection module 134 for the next eligibility list for a sooner appointment. When the patient 20 of interest is selected (i.e., “Select”), at step 658 the payment module 136 completes the pre-loaded payment from the patient 20. At step 660, the patient 20 arrives at the appointment at the sooner scheduled appointment. At step 662 the patient 20 is fulfilling an unfulfilled appointment and, in some examples, the patient 20 can be seen within minutes of arrival at the provider 30. At step 664, the patient 20 completes the sooner appointment (as performed by the provider 30), and therefore has received the procedure sooner than originally scheduled, and has waited less time.

Referring again to step 660, in an example, the appointment application 130 determines the best or reasonably suitable time prior to the appointment time for the patient 20 to leave in order for that patient 20 to successfully attend the appointment, and sends a communication to the patient 20 at that time. For example, the appointment application 130 uses the calendar information, the location information (e.g. GPS tracking or expected location from the calendar information), weather information, traffic information, or other information, to determine the time to leave. The appointment application 130 can determine the best time for the patient 20 to leave at the beginning of the day of the appointment and updated in real time throughout the day. In another example, the best time to leave is not too early, therefore reducing potential wasted waiting time by the patient 20. In another example, a machine learning model is used to determine the best time for the patient 20 to leave.

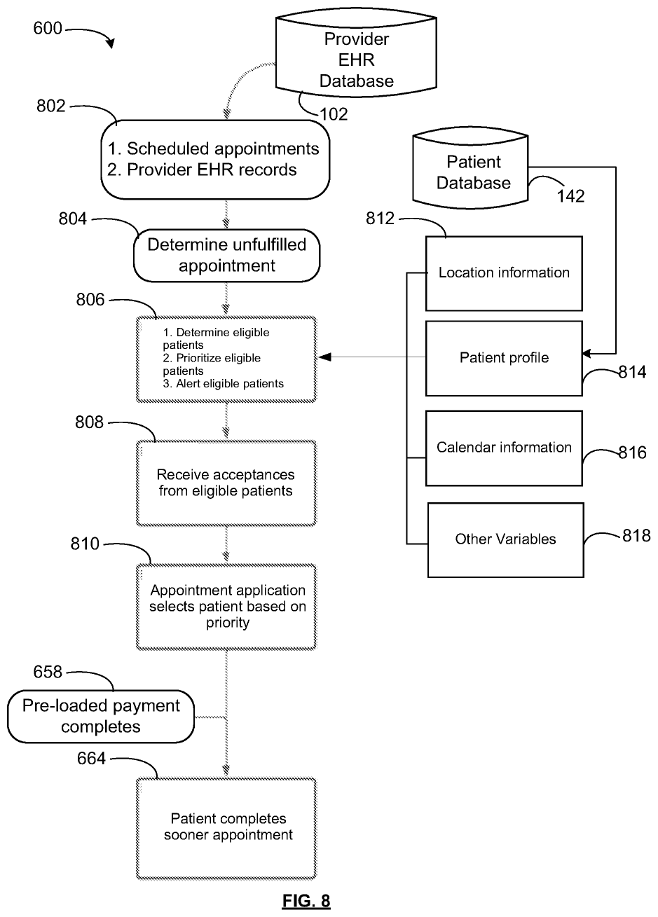

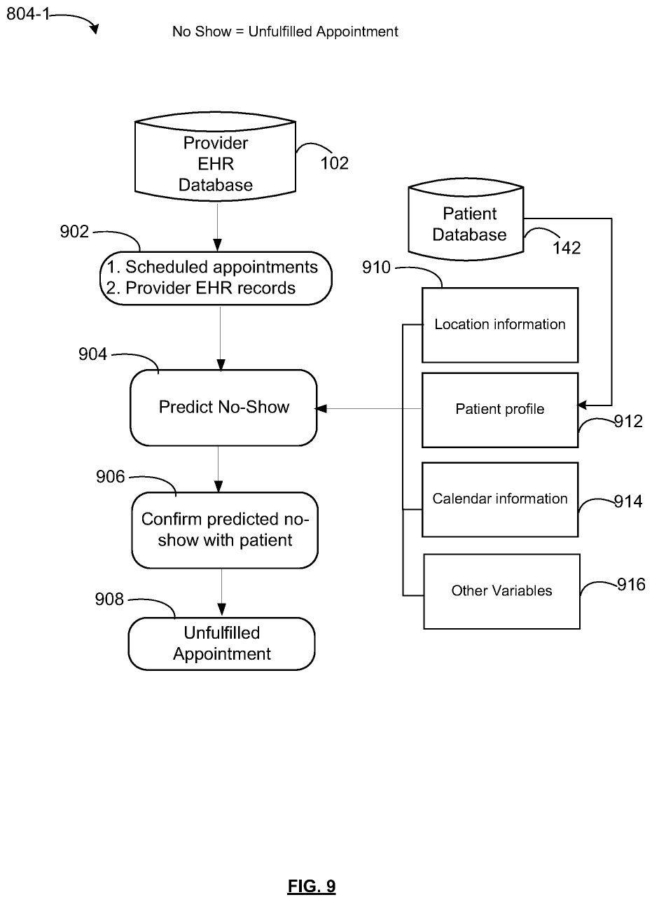

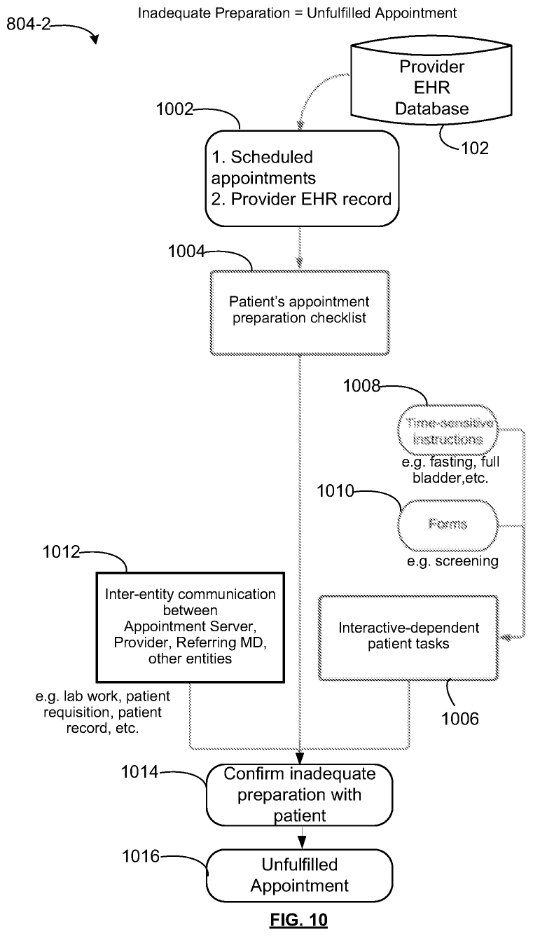

FIG. 8 illustrates a detailed example of the backend of the method 600 as performed by the appointment application 130, in accordance with an example embodiment. The method 600 details an example of how to determine and prioritize eligible patients to fill the unfulfilled timeslot. at step 802, the appointment application 130 receives, from the provider EHR database 102: i) the schedule (scheduled appointments) of the provider 30, and ii) the provider EHR records. At step 804, the appointment application 130 determines that one of the scheduled appointments has an unfulfilled timeslot (unfulfilled appointment). In another example, the appointment application 130 predicts the unfulfilled timeslot using the unfulfilled appointment predictor model, described in greater detail below with respect to FIG. 9. In an example, the appointment application 130 uses the unfulfilled appointment predictor module 132 to determine the unfulfilled timeslot. In an example, the appointment application 130 determines the unfulfilled timeslot by determining inadequate preparation, described in greater detail with below respect to FIG. 10. In another example of step 804, the appointment application 130 simply receives a cancellation notification from the provider EHR database 102 or directly from the patient 20 (e.g., via the device 108). In some examples, the patient 20 having the original scheduled appointment is rescheduled by the appointment application 130 or by the provider EHR server 112.

At step 806, the patient selection module 134 of the appointment application 130 receives information in order to: i) determine eligible patients to fill the unfulfilled timeslot, ii) prioritize the eligible patients, and iii) alert the eligible patients. In various examples, the information includes: location information 812, patient profile 814 from the patient database 142, calendar information 816, and other variables 818. In various examples, the information also includes the provider EHR records from the provider EHR database 102.

Still referring to step 806, the patient selection module 134 can be trained or use various rules to i) determine eligible patients to fill the unfulfilled timeslot. For example, the patient 20 may be ineligible to fill the unfulfilled timeslot due to another appointment for the unfulfilled timeslot, as indicated by their calendar information 816 (from their calendar application 407). In some instances, the patient 20 may not be in the geographical vicinity of the provider 30 at the time of the unfulfilled timeslot, in view of their location information 812 or the calendar information. Similarly, the location information 812 and the calendar information can be used to determine that the patient 20 is available (no scheduling conflicts) and within a suitable location of the provider 30. The time length of the procedure of each patient 20 is another factor. If the time length of the procedure of a patient 20 is within the length of the unfulfilled timeslot, then that patient 20 may be considered eligible.

In another example, the unfulfilled timeslot may have been determined (at step 804) on the same day as the unfulfilled timeslot. In such an example, the location information from the GPS application 406 of each patient 20 is used to determine whether that patient 20 would be able to attend the unfulfilled timeslot, and if so that patient 20 could be considered an eligible patient for filling the unfulfilled timeslot. Other variables 818 can include weather information and traffic information. For example, the patient selection module 134 can calculate a travel time for the patient 20, based on their information location, the weather information and the traffic information (and other information). The location information can also be obtained from the calendar application 407, to predict whether the patient 20 will be in the vicinity at the time of the unfulfilled timeslot and the location of the provider 30.

Still referring to step 806, the patient selection module 134 can be trained or use various rules to ii) prioritize the eligible patients. For example, the patient 20 was previously eligible but not selected by the appointment application 130 for a previous unfilled appointment. In such a scenario, the patient selection module 134 generates a priority in which the patient 20 has a higher priority. Another example is that the patient selection module 134 can use the patient health status from the patient profile 814 or the provider EHR record, in which a patient 20 with a worsened health status may be deemed by the patient selection module 134 to have a higher priority.

Another example of higher priority is the probability that the patient 20 would be able to attend the unfulfilled appointment. For example, the unfulfilled timeslot may have been determined (at step 804) on the same day as the unfulfilled timeslot. A patient 20 can be determined as having a higher priority based on the travel time for the patient 20, using their information location, the weather information and the traffic information (and other information).

In an example, the patient selection module 134 includes the patient selection model. In an example, the patient selection model is trained to optimize the probability that the patient 20 being selected will fill the unfulfilled timeslot. For example, the same or similar criteria as the unfulfilled appointment predictor module 132 can be used. In another example, the patient selection model is trained to optimize overall patient health of all patients 20 waiting for a sooner appointment, based on the patient health status of the patients 20, and trained to minimize health incidents.

Still referring to step 806, at step iii) the patient selection module 134 alerts the eligible patients. At step 808, the patient selection module 134 receives patient acceptances (and sometimes rejections) in response to the alerts. At step 810, the patient selection module 134 selects one or more patients 20 to fill the unfulfilled timeslot, based on the priority generated at step 806. At step 658, the payment module 136 completes the pre-loaded payment. At step 664, the patient completes the sooner appointment (e.g., a procedure as performed by the provider 30).

FIG. 9 illustrates an example of the process 804-1 of determining an unfulfilled appointment, by way of a no-show, in accordance with an example embodiment. For example, the unfulfilled appointment predictor model predicts a no-show for a scheduled appointment. At step 902, the appointment application 130 receives, from the provider EHR database 102: i) the schedule (scheduled appointments) of the provider 30, and ii) the provider EHR records. At step 904, the unfulfilled appointment predictor model predicts a no-show. At step 904, the unfulfilled appointment predictor model receives information in order to predict the no-show. In various examples, the information includes: location information 910, patient profile 912 from the patient database 142, calendar information 914, and other variables 916. Other variables 916 can include weather information and traffic information. In various examples, the information also includes the provider EHR records from the provider EHR database 102. In an example, the unfulfilled appointment predictor model is trained to maximize the accuracy of prediction of no-show appointments, in order to minimize the occurrence of no-show appointments.

In another example, the unfulfilled appointment predictor model infers a probability that a particular timeslot could be unfulfilled. The real-time probability is transmitted from the appointment application 130 to the device 108 for display through the frontend appointment application 408. Accordingly, users such as patients 20, providers 30, and referring MDs 40 can monitor likelihood of an unfulfilled timeslot and be on standby for when a notification is transmitted.

Still referring to step 904, the unfulfilled appointment predictor module 132 can be trained or use various rules to predict a no-show. For example, when a patient 20 has a scheduled appointment and the location information or the calendar information (or both) suggests or has a higher probability that the patient 20 will not be able to arrive at the scheduled appointment, the appointment application 130 can infer that the scheduled appointment is a no-show and has an unfulfilled timeslot. Other variables 916 can be used for the inference by the appointment application 130, such as weather, traffic, etc.

At step 906, when a no-show is predicted (e.g. above a certain threshold probability), then the appointment application 130 determines that there is a probability of a no-show and confirms with the patient 20. In some examples, the confirmation is a notification to the patient 20 that their scheduled appointment is cancelled. In other examples, the confirmation is a notification to the patient that their scheduled appointment will be cancelled unless the patient 20 sends a response within a deadline. In an example, the deadline is a reasonable amount of time, such as any deadline between 1 day and 1 week.

At step 908, when the patient 20 confirms that they will not attend or the deadline expires prior to any response, the appointment application 130 determines that the scheduled appointment is an unfulfilled appointment (unfulfilled timeslot) due to the no-show of the patient 20.

FIG. 10 illustrates an example of the process 804-2 of determining an unfulfilled appointment, by way of inadequate preparation, in accordance with an example embodiment. At step 1002, the appointment application 130 receives, from the provider EHR database 102: i) the schedule (scheduled appointments) of the provider 30, and ii) the provider EHR records. At step 1004, the appointment application 130 determines (e.g., generates or otherwise receives from the provider 30 or referring MD 40) a patient appointment preparation checklist, and sends to the patient 20. The patient 20 may take steps to prepare and fulfill the patient appointment preparation checklist, and input into the device 108. At step 1008, the patient 20 may perform (and input into the device 108) time-sensitive instructions, such as fasting, full bladder, etc. At step 1010, the patient may input into one or more forms into the device 108. At step 1006, the appointment application 130 determines whether the interactive-dependent patient tasks are completed. At step 1012, inter-entity communication may need to be completed between the appointment server 100, the referring MDs 40, the providers 30, and other entities such as labs. For example, information may need to be transferred prior to the scheduled appointment, such as lab work, patient requisition, patient record, etc.

At step 1014, if any of the steps of the process 804-2 fail, then the appointment application 130 determines that there is inadequate preparation and confirms with the patient 20. In some examples, the confirmation is a notification to the patient 20 that their scheduled appointment is cancelled. In other examples, the confirmation is a notification to the patient that their scheduled appointment will be cancelled unless any of the deficiencies are remedied within a deadline. In an example, the deadline is a reasonable amount of time to fulfill the deficiencies (which may include lab work), such as any deadline between 2 days and 1 week. At step 1016, the appointment application 130 determines that the scheduled appointment is an unfulfilled appointment (unfulfilled timeslot).

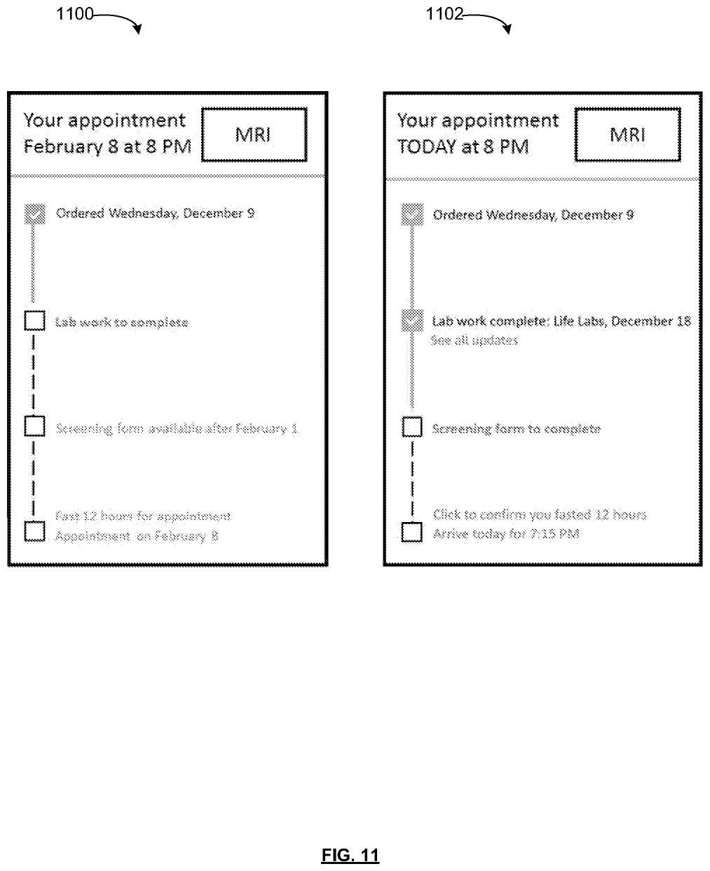

FIG. 11 illustrates a first screen 1100 and a second screen 1102 of the device 108 of a patient 20, illustrating the preparation required by the patient 20. The second screen 1102 is subsequent to the first screen 1100. The first screen 1100 and the second screen 1102 illustrate timelines, in which, a bolder thicker solid line means that the particular activity has been completed, and a thinner dashed line means that the particular activity has not yet been completed. The first screen 1100 includes a timeline that includes activities for preparation by the patient 20. In the illustrated example, the example timeline includes: i) order date, ii) lab work to complete, iii) screening form, iv) and fasting 12 hours prior to appointment. The first screen 1100 illustrates that i) order date has been completed. The second screen 1102 illustrates further that ii) lab work has been completed. If any of the tasks are not completed by the deadline, then the appointment application 130 determines that there is inadequate preparation and confirms with the patient 20 (e.g. step 1014 in FIG. 10).

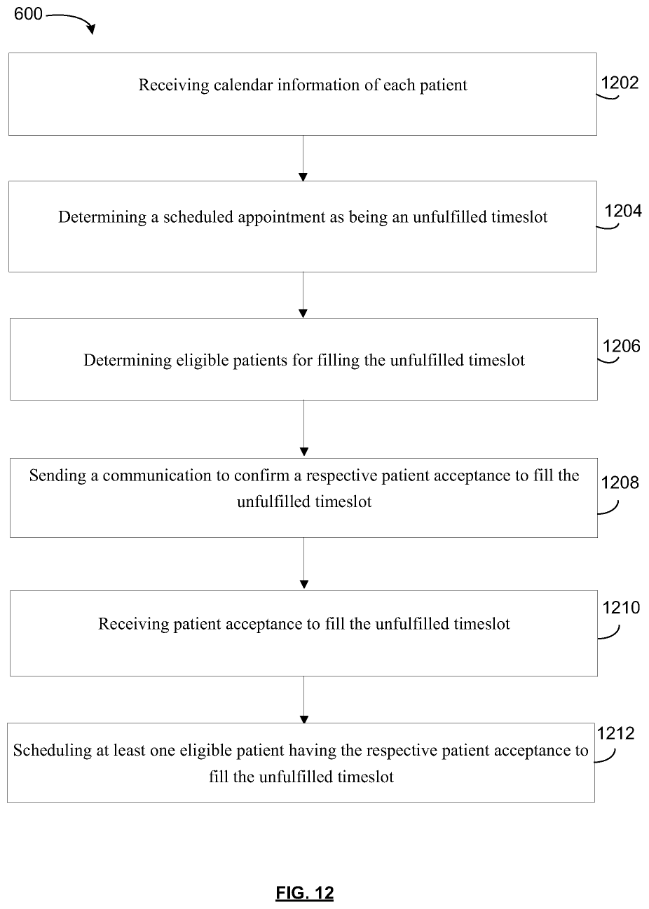

FIG. 12 illustrates another example of the method 600 of appointment scheduling performed by the appointment application 130, in accordance with an example embodiment. At step 1202, the appointment application 130 receives, from the respective device 108 of at least one patient 20, calendar information of that patient 20 from the calendar application 407 of that device 108.

At step 1204, the appointment application 130 determines a scheduled appointment as being an unfulfilled timeslot. For example, the unfulfilled appointment predictor model predicts that the scheduled appointment will be unfulfilled due to a no-show or inadequate preparation of the patient 20.

At step 1206, the appointment application 130 determines at least one patient 10 as being an eligible patient for filling the unfulfilled timeslot based on the calendar information of the respective device 108.

At step 1208, the appointment application 130 sends a communication to the respective device 108 of at least one of the eligible patients 20 to confirm a respective patient acceptance to fill the unfulfilled timeslot. In an example, the communication includes a deadline as to when patient acceptances are required. If the response from a patient 20 is not received by the deadline, then there is no patient acceptance for that patient 20. In an example, the deadline is only a short period of time, such as 5 minutes or 1 hour. In another example, the deadline is a longer period such as from 1 day to 1 week.

At step 1210, the appointment application 130 receives the respective patient acceptance from at least one eligible patient 20 to fill the unfulfilled timeslot. For example, the patient acceptances are received prior to the deadline.

At step 1212, the appointment application 130 schedules at least one eligible patient 20 having the respective patient acceptance to fill the unfulfilled timeslot. These patients 20 now have a sooner appointment than their originally scheduled appointments.

In other examples of the method 600, the appointment application 130 determines a priority of at least one of the eligible patients; and selects, based on the priority, the at least one eligible patient to fill the unfulfilled timeslot for the scheduling.

It can be appreciated that the steps of the method 600 of appointment scheduling are performed in a different order. For example, the priority of the patients 20 can be determined for all patients 20 who wish to have a sooner appointment, before the eligibility of the patients 20 to fill the unfulfilled timeslot at step 1206. Those eligible patients therefore have a priority order.

In another example, the priority of the patients 20 is determined after receiving the respective patient 20 acceptance to fill the unfulfilled timeslot. In another example, the priority of the patients 20 is first determined from all patients 20 who wish to have a sooner appointment, and then a cutoff threshold of the top priority patients 20 are then used to define the eligible patients at step 1206. Those eligible patients therefore have a priority order.

Examples of the procedures for the scheduled appointments include specialist care from a medical specialist, such as a cardiologist, allergist, urologist/gynecologist or psychiatrist, for a diagnosis or consultation. Examples of the procedures for the scheduled appointments include non-emergency surgery, including any scheduled or planned non-emergency surgery, such as cardiac surgery, joint surgery (e.g., knee or hip), caesarean sections and cataract surgery. Examples of the procedures for the scheduled appointments include selected diagnostic tests are tests that include magnetic resonance imaging (MRI) scans, computerized tomography scans (CT scans), and angiographies provided in non-emergency situations.

Examples of the procedures for the scheduled appointments include: bacteriological laboratories, diagnostic; bacteriological laboratories, medical; biological laboratories; blood analysis laboratories; dental laboratories, X-ray; dental laboratory, analysis and diagnostic; health laboratory (e.g., cytology, mycology, parasitology, toxicology); laboratories, biological, medical, X-ray (picture and treatment); laboratories, combined medical and radiological; laboratories, dental X-ray; laboratory services, medical; medical and diagnostic laboratories; medical laboratories (clinical); medical pathology laboratories; medical X-ray services; radiological laboratory services, medical; testing laboratories, medical, analytic or diagnostic; urinalysis laboratories; X-ray laboratories, medical; X-ray, MRI, computerized axial tomography (CAT)/CT scan, ultrasound and other medical imaging centers with medical doctors; X-ray, MRI, CAT/CT scan, ultrasound and other medical imaging centers without medical doctors.

In the example embodiments, it should be understood that the disclosed system, appointment server, and method of appointment scheduling may be implemented in other manners. For example, the described appointment server is merely an example embodiment. For example, the unit division is merely logical function division and may be other division in actual implementation. For example, a plurality of units or components may be combined or integrated into another system, or some features may be ignored or not performed. In addition, the displayed or discussed mutual couplings or direct couplings or communication connections may be implemented by using some interfaces. The indirect couplings or communication connections between the units may be implemented in electronic, mechanical, or other forms.

The units described as separate parts may or may not be physically separate, and parts displayed as units may or may not be physical units, may be located in one position, or may be distributed on a plurality of network units. Some or all of the units may be selected according to actual requirements to achieve the objectives of the solutions of the embodiments.

In addition, functional units in the example embodiments may be integrated into one processing unit, or each of the units may exist alone physically, or two or more units are integrated into one unit.

When the functions are implemented in the form of a software functional unit and sold or used as an independent product, the functions may be stored in a computer-readable storage medium. Based on such an understanding, the technical solutions of example embodiments may be implemented in a form of a software product. The software product is stored in a storage medium, and includes several instructions for instructing a computer device (which may be a personal computer, a server, or a network device) to perform all or some of the steps of the methods described in the example embodiments. The foregoing storage medium includes any medium that can store program code, such as a Universal Serial Bus (USB) flash drive, a removable hard disk, a read-only memory (Read-Only Memory, ROM), a random access memory (Random Access Memory, RAM), a magnetic disk, or an optical disc.

In the described methods or block diagrams, the boxes may represent events, steps, functions, processes, modules, messages, and/or state-based operations, etc. While some of the above examples have been described as occurring in a particular order, it will be appreciated by persons skilled in the art that some of the steps or processes may be performed in a different order provided that the result of the changed order of any given step will not prevent or impair the occurrence of subsequent steps. Furthermore, some of the messages or steps described above may be removed or combined in other embodiments, and some of the messages or steps described above may be separated into a number of sub-messages or sub-steps in other embodiments. Even further, some or all of the steps may be repeated, as necessary. Elements described as methods or steps similarly apply to systems or subcomponents, and vice-versa. Reference to such words as “sending” or “receiving” could be interchanged depending on the perspective of the particular device.

The above discussed embodiments are considered to be illustrative and not restrictive. Example embodiments described as methods would similarly apply to systems, and vice-versa.

The various embodiments presented above are merely examples and are in no way meant to limit the scope of this disclosure. Variations of the innovations described herein will be apparent to persons of ordinary skill in the art, such variations being within the intended scope of the present disclosure. In particular, features from one or more of the above-described embodiments may be selected to create alternative embodiments comprises of a sub-combination of features which may not be explicitly described above. In addition, features from one or more of the above-described embodiments may be selected and combined to create alternative embodiments comprised of a combination of features which may not be explicitly described above. Features suitable for such combinations and sub-combinations would be readily apparent to persons skilled in the art upon review of the present disclosure as a whole. The subject matter described herein intends to cover all suitable changes in technology.

1. A method of appointment scheduling, comprising:
receiving, from a respective device of at least one patient, calendar information of that patient from a calendar application of that device;
determining a scheduled appointment as being an unfulfilled timeslot;
determining at least one patient as being an eligible patient for filling the unfulfilled timeslot based on the calendar information of the respective device;
sending a communication to the respective device of at least one of the eligible patients to confirm a respective patient acceptance to fill the unfulfilled timeslot;
receiving the respective patient acceptance from at least one eligible patient to fill the unfulfilled timeslot; and
scheduling at least one eligible patient having the respective patient acceptance to fill the unfulfilled timeslot.

2. The method as claimed in claim 1, further comprising:
determining a priority of at least one of the eligible patients; and
selecting, based on the priority, the at least one eligible patient to fill the unfulfilled timeslot for the scheduling.

3. The method as claimed in claim 2, wherein the priority is based on a respective patient electronic health record of the patient.

4. The method as claimed in claim 3, wherein the priority is based on a respective health status of the patient from the respective patient electronic health record.

5. The method as claimed in claim 2, wherein the priority of one of the patients increases when that patient had sent a previous patient acceptance and was not scheduled to fill a previous unfulfilled timeslot.

6. The method as claimed in claim 1, wherein the communication includes a deadline, wherein the receiving the respective patient acceptance is within the deadline in order for the scheduling to be authorized.

7. The method as claimed in claim 1, wherein the determining at least one patient as being the eligible patient is based on a time length of the unfulfilled timeslot and a respective time length of procedure for the eligible patient.

8. The method as claimed in claim 1, further comprising receiving, from the respective device of at least one patient, location information of that patient;
wherein the determining at least one patient as being the eligible patient is further based on the location information of that patient.

9. The method as claimed in claim 8, wherein the device has a Global Positioning System, wherein the location information is obtained from the Global Positioning System of the respective device of the patient.

10. The method as claimed in claim 8, wherein the location information is obtained from the calendar application of the respective device of the patient.

11. The method as claimed in claim 8, wherein the location information is obtained from an input address of that patient.

12. The method as claimed in claim 1, wherein the unfulfilled timeslot is from a patient originally in the unfulfilled timeslot, wherein the determining the scheduled appointment as being the unfulfilled timeslot is determined from the calendar information of the calendar application of the respective device of that patient originally in the unfulfilled timeslot.

13. The method as claimed in claim 1, further comprising receiving, from the respective device of at least one patient, location information of that patient;
wherein the unfulfilled timeslot is from a patient originally in the unfulfilled timeslot; wherein the determining the scheduled appointment as being the unfulfilled timeslot is based on the location information of that patient originally in the unfulfilled timeslot.

14. The method as claimed in claim 13, wherein the device has a Global Positioning System, wherein the location information is based from the Global Positioning System of the respective device of the patient.

15. The method as claimed in claim 13, wherein the location information is obtained from the calendar application of the respective device of the patient.

16. The method as claimed in claim 13, wherein the location information is obtained from an input address of that patient.

17. The method as claimed in claim 1, wherein the unfulfilled timeslot is from a patient originally in the unfulfilled timeslot, wherein the determining the schedule appointment as being the unfulfilled timeslot is based on the calendar information of that patient originally in the unfulfilled timeslot.

18. The method as claimed in claim 1, further comprising:
receiving a respective pre-loaded payment from at least one patient; and
applying the respective pre-loaded payment as a consequence to the scheduling at least one eligible patient.

19. The method as claimed in claim 1, wherein the unfulfilled timeslot is from a patient originally in the unfulfilled timeslot, wherein the determining the unfulfilled timeslot is due to a no-show of that patient originally in the unfulfilled timeslot.

20. The method as claimed in claim 1, wherein the unfulfilled timeslot is from a patient originally in the unfulfilled timeslot, wherein the determining the unfulfilled timeslot is due to inadequate preparation of that patient originally in the unfulfilled timeslot.

21. The method as claimed in claim 1, wherein the determining the unfulfilled timeslot is performed using an unfulfilled appointment predictor model.

22. The method as claimed in claim 1, further comprising selecting, using a patient selection model, the at least one eligible patient to fill the unfulfilled timeslot for the scheduling.

23. The method as claimed in claim 1, wherein the unfulfilled timeslot is from a patient originally in the unfulfilled timeslot, wherein the unfulfilled timeslot is due to a no-show of that patient originally in the unfulfilled timeslot.

24. The method as claimed in claim 1, wherein the scheduled appointment is stored in a provider electronic health record database.

25. The method as claimed in claim 1, further comprising receiving a request from at least one patient to be one of the eligible patients.

26. The method as claimed in claim 1, wherein the unfulfilled timeslot is from a patient originally in the unfulfilled timeslot, further comprising rescheduling the patient originally in the unfulfilled timeslot.

27. The method as claimed in claim 1, wherein each patient has a respective scheduled appointment, further comprising receiving from at least one patient a request to be the eligible patient for filling the unfulfilled timeslot.

28. The method as claimed in claim 27, wherein the request includes a referral from a referring medical doctor.

29. The method as claimed in claim 1, wherein the at least one eligible patient scheduled to fill the unfulfilled timeslot has a respective scheduled appointment with a first provider, wherein the unfulfilled timeslot is with a second provider different than the first provider.

30. The method as claimed in claim 1, wherein the unfulfilled timeslot is with a provider, wherein the method is performed by a processor operated by a third party from the provider, wherein the method is performed by the processor for multiple different providers including the provider.

31. A server, comprising:
a processor; and
memory containing instructions which, when executed by a processor, cause the processor to:
receive, from a respective device of at least one patient, calendar information of that patient from a calendar application of that device,
determine a scheduled appointment as being an unfulfilled timeslot,
determine at least one patient as being an eligible patient for filling the unfulfilled timeslot based on the calendar information of the respective device,
send a communication to the respective device of at least one of the eligible patients to confirm a respective patient acceptance to fill the unfulfilled timeslot,
receive the respective patient acceptance from at least one eligible patient to fill the unfulfilled timeslot, and
schedule at least one eligible patient having the respective patient acceptance to fill the unfulfilled timeslot.

32. A non-transitory memory containing instructions which, when executed by a processor, cause the processor to perform appointment scheduling, the instructions comprising:
instructions for receiving, from a respective device of at least one patient, calendar information of that patient from a calendar application of that device;
instructions for determining a scheduled appointment as being an unfulfilled timeslot;
instructions for determining at least one patient as being an eligible patient for filling the unfulfilled timeslot based on the calendar information of the respective device;
instructions for sending a communication to the respective device of at least one of the eligible patients to confirm a respective patient acceptance to fill the unfulfilled timeslot;
receiving the respective patient acceptance from at least one eligible patient to fill the unfulfilled timeslot; and
instructions for scheduling at least one eligible patient having the respective patient acceptance to fill the unfulfilled timeslot.

33. (canceled)

34. (canceled)
